(19) United States
(12) Patent Application Publication
(10) Pub. No.: US 2026/0308501 A1
(43) Pub. Date: Oct. 8, 2026

(54) PERFUSED CELL CULTURING SYSTEMS AND USES THEREOF

(71) Applicants: Jason P. Gleghorn, Newark, DE (US); Katherine Nelson, Newark, MD (US); Daniel J. Minahan, Newark, DE (US)

(72) Inventors: Jason P. Gleghorn, Newark, DE (US); Katherine Nelson, Newark, MD (US); Daniel J. Minahan, Newark, DE (US)

(73) Assignee: University of Delaware, Newark, DE (US)

(21) Appl. No.: 19/482,688

(22) PCT Filed: May 10, 2024
(86) PCT No.: PCT/US2024/028828

§ 371 Date: Nov. 7, 2025

Related U.S. Application Data
(60) Provisional application No. 63/465,386, filed on May 10, 2023.

(51) Int. Cl. C12M 3/00; C12M 1/00; C12M 1/12; C12N 5/071; C12N 5/073

(52) U.S. Cl. CPC C12M 23/42; C12M 23/38; C12M 25/02; C12M 29/10; C12N 5/0605; C12N 5/069

(57) ABSTRACT

The present invention provides a system for culturing cells. The system comprises a cassette, an insert having a cell culture platform, and cultured cells attached to or in the cell culture platform. Where the insert is in the cassette, the cultured cells may be exposed to a flowing fluid with a wall shear stress at, for example, 0.25-75 dyne/cm². The cultured cells may be seeded and grown into, for example, a monolayer attached to the cell culture platform in the insert outside the cassette before the insert is placed in the cassette and the cultured cells are exposed to a flowing fluid. The insert may be removed from the cassette while the cultured cells remain attached to the cell culture platform in the insert. Also provided are a method for culturing cells in the system and a method for preparing the system.

### CROSS-REFERENCE TO RELATED APPLICATION

This application claims priority to U.S. Provisional Application No. 63/465,386, filed May 10, 2023, and the contents of which are incorporated herein by reference in their entireties for all purposes.

### REFERENCE TO U.S. GOVERNMENT SUPPORT

This invention was made with government support under grant number 5U19AI158930 awarded by National Institutes of Health. The United States has certain rights in the invention.

### FIELD OF THE INVENTION

The invention relates to modular systems for culturing cells on perfused organ-on-a-chip platforms.

### BACKGROUND OF THE INVENTION

Organ-on-a-chip (OOC) technologies have advanced tissue models to recapitulate in vivo physiology more accurately. These tissue models have been well established as valuable in vitro tools for both basic and translational science. Inherently built into the models is the ability to control fluid flow, and therefore shear stress, applied to the cells, which increases physiologic relevance, compared to standard static 2D or 3D cultures. Shear stress is an important stimulus in many tissue types (kidney and lung development, stem cell differentiation), making OOC models invaluable in the progress of biomedical sciences.

Unfortunately, few labs have access to facilities capable of fabricating these devices. End users often attempt to adapt complex published models to meet their needs; however, the complexity and nuance of the protocols often results in poor reproducibility. It then becomes paramount to have expertise and the materials necessary to collect and interpret quality data. Typically, OOC models are fabricated in a cleanroom with expensive, technical equipment. These methods, like soft lithography, while reproducible and precise, limit the use of microfluidic-based systems to labs with funding, space, and technical expertise in microfluidic fabrication. Conversely, commercial OOC systems have shown promise in migrating knowledge into non-expert users' hands in a reproducible manner, but at a large cost.

Customization of standard OOC systems highlight their applicability to study cell and tissue interactions, but they are limited in their design. Often, the fluidic channels are irreversibly sealed, making any changes impossible without fabricating and seeding a new device, wasting time and precious samples. In addition, the dynamic fluid flows inherent to OOC models supply shear forces and steady nutrient diffusion to maintain the cell culture, however, working with such small volumes when seeding the cells can often lead to failure. There is a need for a simple to use, inexpensive OOC system that overcomes these hurdles. A modular OOC system would allow for easier cell seeding and remove the irreversible nature of current devices, allowing for additional sampling, dosing, or imaging of the cell sample. In addition, a modular device design can easily be used with different materials and designs can be quickly updated depending on the properties required for an experiment giving researchers more control of their experiments.

Rapid prototyping techniques such as 3D printing, laser cutting, and other smart craft cutting equipment have been tools that expanded manufacturing accessibility due to their relative low-cost and ease of use. Laser cutting provides fast manufacturing and precise geometries at low-cost. A laser cut microfluidic cell culture system has been fabricated and shown to enable fluid flow and cell culture, which provides labs with access to a clean-room free method to design and create their own system. However, this, and other similar systems, are still irreversibly sealed which limits the types and number of assays that can be run on a single device. Designing reusable components and tolerant cell processing assays in a rapid prototyped system will allow for multiple experiments to be run using one base system and fewer disposable components, further lowering costs and ease of use.

Recently, the “FDA Modernization Act 2.0” removed the requirement of testing in animal models and now promotes substitutes in pharmacologic research such as OOC technologies. This yields a greater need to democratize these technologies.

### SUMMARY OF THE INVENTION

The present invention relates to a system for culturing cells. The inventors have surprisingly discovered a modular organ-on-a-chip system in which the cultured cells are exposed to a flowing fluid.

The present invention provides a system for culturing cells. The system comprises:
(a) a cassette comprising a lid and a base, wherein the lid comprises a top channel inlet, a top channel outlet, a bottom channel inlet and a bottom channel outlet;
(b) an insert in the cassette, wherein the insert is between the lid and the base, wherein the insert comprises a top layer having a top channel, a bottom layer having a bottom channel, and a cell culture platform having a top platform surface in contact with the top channel and a bottom platform surface in contact with the bottom channel, wherein the cell culture platform is selected from the group consisting of a vitrified membrane, a hydrated gel, a semi-permeable membrane, and a combination thereof, wherein the top channel is sealed by the lid and the top platform surface and connected with the top channel inlet and the top channel outlet, wherein a top flowing fluid moves to create a wall shear stress in the top channel of 0.25-75 dyne/cm² through the top channel inlet, the top channel, and the top channel outlet; wherein the bottom channel is sealed by the base and the bottom platform surface, and connected with the bottom channel inlet and the bottom channel outlet, wherein a bottom flowing fluid moves to create a wall shear stress in the bottom channel of 0.25-75 dyne/cm² through the bottom channel inlet, the bottom channel, and the bottom channel outlet, and
(c) first cultured cells, wherein the first cultured cells are attached to the top platform surface and exposed directly to the top flowing fluid, are attached to the bottom platform surface and exposed directly to the bottom flowing fluid, or are in the cell culture platform and exposed indirectly to the top flowing fluid and indirectly to the bottom flowing fluid.

The vitrified membrane may have a thickness of 2-50 μm. The culture platform may consist of the vitrified membrane. The system may further comprise second cultured cells. The vitrified membrane may comprise collagen-I, the first cultured cells may be BeWo b30 cells, and the second cultured cells may be human umbilical vein endothelial cells (HUVECs).

The hydrated gel may have a thickness of 50-1500 μm. The cell culture platform may consist of the hydrated gel. The system may further comprise second cultured cells. The system may further comprise third cultured cells. The hydrated gel may be a hydrogel comprising collagen-I or decellularized placental extracellular matrix (ECM), the first cultured cells may be BJ fibroblasts, the second cultured cells may be BeWo b30 cells, and the third cultured cells may be HUVECs.

The cell culture platform may consist of the semi-permeable membrane having a first semi-permeable membrane surface and a second semi-permeable membrane surface, and the first culture cells may be attached to the first semi-permeable membrane surface. The system may further comprise second cultured cells attached to the second semi-permeable membrane surface.

The culture platform may consist of the hydrated gel having a first gel surface and a second gel surface and the semi-permeable membrane having a first semi-permeable membrane surface and a second semi-permeable membrane surface, and the second gel surface and the first semi-permeable membrane surface are in direct contact. The system may further comprise second cultured cells. The system may further comprise third cultured cells.

The vitrified membrane may comprise first ECM proteins and the first cultured cells may be attached to a surface of the vitrified membrane.

The hydrated gel may comprise second ECM proteins and the first cultured cells may be in the hydrated gel.

The semi-permeable membrane may have a semi-permeable membrane surface coated with third ECM proteins and the first cultured cells may be attached to the coated semi-permeable membrane surface.

The insert may further comprise an adhesive film.

The lid may further comprise a glass surface.

The top channel inlet may be connected with a first perfusion pump. The bottom channel inlet may be connected with a second perfusion pump. The first perfusion pump and the second perfusion pump may be the same.

The system may exclude polydimethylsiloxane (PDMS).

The top flowing fluid may comprise a top agent selected from the group consisting of media, cells, blood, microbiota, pharmacological agents, normoxic air, anoxic air and a combination. The top agent may migrate from the top channel into the cell culture platform. The top agent may migrate from the top fluidic channel through the cell culture platform and into the bottom channel.

The bottom flowing fluid may comprise a top agent selected from the group consisting of media, cells, blood, microbiota, pharmacological agents, normoxic air, anoxic air and a combination. The bottom agent may migrate from the top channel into the cell culture platform. The bottom agent may migrate from the top fluidic channel through the cell culture platform and into the bottom channel.

For each system of the present invention, a method for growing cells is provided. The cell growing method comprises incubating the first cultured cells in the system. The first cultured cells may be in a monolayer attached to the top or bottom platform surface.

The cell growing method may further comprise (i) seeding cells onto the top platform surface or the bottom platform surface in the insert outside of the cassette, whereby the seeded cells are attached to the top or bottom platform surface; (ii) growing the attached seeded cells, wherein the insert having the first cultured cells in a monolayer attached to the top or bottom platform surface is prepared; and (iii) placing the insert from step (ii) in the cassette to establish fluid flow.

The cell growing method may further comprise removing the insert from the cassette. The cell growing method may further comprise characterizing the first cultured cells remaining attached to the cell culture platform in the insert after the removal.

The present invention further provides a method for preparing a system for culturing cells. The preparation method comprises:
(a) fabricating a cassette with a lid and a base, wherein the lid comprises a top channel inlet, a top channel outlet, a bottom channel inlet and a bottom channel outlet;
(b) providing a cell culture platform having a top platform surface and a bottom platform surface, wherein the cell culture platform is selected from the group consisting of a vitrified membrane comprising first extracellular matrix (ECM) proteins, a hydrated gel comprising second ECM proteins, a semi-permeable membrane, and a combination thereof;
(c) assembling an insert comprising the cell culture platform, a top layer having a top channel in contact with the top platform surface, and a bottom layer having a bottom channel in contact with the bottom platform surface;
(d) placing the insert in the cassette, wherein the insert is between the lid and the base, whereby the top channel is sealed by the lid and the top platform surface and connected with the top channel inlet and the top channel outlet, and the bottom channel is sealed by the base and the bottom platform and connected with the bottom channel inlet and the bottom channel outlet;
(e) perfusing a top flowing fluid at a top channel wall shear stress of 0.25-75 dyne/cm² through the top channel inlet, the top channel, and the top channel outlet;
and
(f) perfusing a bottom flowing fluid at a bottom channel wall shear stress 0.25-75 dyne/cm² through the bottom channel inlet, the bottom channel, and the bottom channel outlet.

According to the preparation method, first cultured cells may be in a monolayer attached to the top platform surface and exposed directly to the top channel and the top flowing fluid or in a monolayer attached to the bottom platform surface and exposed directly to the bottom channel and the bottom flowing fluid.

The cell growing method may further comprise (i) seeding cells onto the top platform surface or the bottom platform surface in the insert outside of the cassette, whereby the seeded cells are attached to the top or bottom platform surface; and (ii) growing the attached seeded cells, whereby the insert having the first cultured cells in a monolayer attached to the top or bottom platform surface is prepared.

According to the preparation method, the first cultured cells may be in the cell culture platform and exposed indirectly to the top flowing fluid and indirectly to the bottom flowing fluid. The insert may further comprise a third layer between the top layer and the bottom layer.

### BRIEF DESCRIPTION OF THE DRAWINGS

FIGS. 1A-D show an insert. (A) Exploded and collapsed views of the insert showcasing, from top to bottom, a top elastomer channel, adhesive layer, cell culture membrane, adhesive layer, and bottom elastomer channel. (B) Highlights methods of using (i) premade elastomer sheets and laminating adhesive to then cut to shape or (ii) using a mold to form the elastomer into the final shape and then applying precut adhesives. Both methods then apply a cell culture membrane. A completed sandwich is made by adding another (i) or (ii) piece upon aligning. The insert is then sterilized to be used for cell culture. (C) Exploded view of the cassette showing the laser cut lid with holes for the fluid ports and screws, adhesive layer, and glass coverslip. The base is comprised of threaded holes to accept the screws. (D) Demonstrating the production of the cassette which requires laser cutting the lid which has a thru slot for visualizing the channels from above, recesses for the adhesive and glass layers, as well as a keyed recess to accept the shape of the insert in one direction for ease of placement and alignment of the fluid ports. The insert is then sandwiched between the lid and base once the lid is assembled. Screws torqued appropriately hold clamping pressure on the insert to create a fluid tight seal ready for cell culture media perfusion.

FIGS. 2A-B show a modular microfluidic system. (A) Exploded view of the modular microfluidic system subunits. Inlet and outlet ports in the cassette lid allow for perfusion across the insert. (B) The assembled insert can be fabricated to the height of channel required by adjusting the elastomer height. Different elastomer heights on the top and bottom can be used depending on the experiment and/or nutrients required for the cell type. Similarly, the width of the overlapping channels can also be adjusted to meet the experimental needs and can differ on top and bottom channels.

FIGS. 3A-C show methods of making vitrified membrane or hydrated extracellular matrix (ECM) gel within an insert.

FIGS. 4A-D show vitrified ECM membranes. SEM of cross section of collagen-I vitrified membrane supported by insert (Ai) and zoomed in (Aii). (B) vitrified membrane made with equal parts collagen-I and fibronectin. (C) Vitrified membrane made with spatially controlled collagen-I (left) and fibronectin (right) with interface at the dotted line (D) SEM of cross section of vitrified membrane made from human placental ECM.

FIGS. 5A-F show statically cultured cells in an insert. (A) Static culture using the device insert. Immunofluorescently stained (B) MDCKs and (C) HUVECs on either side of an insert. (D) Cross-section of the insert showing two cell layers on either side of the insert. Full channel seeding of (E) MDCKs and (F) HUVECs.

FIGS. 6A-E show dynamically cultured cells. (A) Dynamic culture using device insert clamped into the cassette. Immunofluorescently stained(B) MDCKs and (C) HUVECs after culture. Full channel seeding of (D) MDCKs and (E) HUVECs.

FIG. 7 show slices at different heights through a hydrated gel in an insert of the epithelial layer (top left) on the top of the gel, the fibroblasts (top center) embedded within the gel, and the endothelial layer (top right) on the bottom of the gel in a cross-section of the gel (bottom).

FIG. 8 show a cervical model. A2EN endocervical cells with BJ fibroblasts on the other side of the insert (left, center). Circulating immune cells (PMN) may be added to the endothelial channel (right).

### DETAILED DESCRIPTION OF THE INVENTION

The present invention relates to a perfused cell culturing system comprising a cassette, an insert and cultured cells exposed to a flowing fluid. The present invention is based on the inventors' surprising discovery of a modular organ-on-a-chip system, designed for ease of use and manufacturability. A reusable laser-cut acrylic-based system, or “cassette”, may be clamped around a compressible cell culture “insert” to act as a gasket to enable sealed non-leaking fluid flow. The reusable cassette may be designed to include glass coverslips which ensure compatibility with microscopy and may be sterilized for multiple uses. The insert is designed for one time use and may be fabricated to include a commercially available track etched porous membrane or a protein/synthetic polymer scaffold to act as the extra cellular matrix (ECM). The insert allows both thin, vitrified ECM membranes and hydrated ECM gels which may be embedded with cells to be incorporated into the model. These gels are maintained within the confines of the channel within the insert and enable stromal cells to be incorporated within the tissue model. Importantly, both the thin membrane and hydrated gel stromal layers support cell culture. Initiation of cell culture occurs on the insert outside of the cassette in order to allow for parallelization of multiple devices and ease of cell/tissue culture and establishment of the model prior to flow. Once the cell layer is formed, the insert may be clamped within the cassette and flow may be established. This circumvents one of the hurdles of current cell culture microfluidic chips by establishing a monolayer of cells in an easily accessible space rather than within the confined space of a microfluidic device. At defined timepoints or after the experiment, the insert may be removed from the cassette and assayed and placed back within the cassette for continued culture of the system. The channel dimensions may be adjusted to the required nutrient load. The system of the present invention is easy to fabricate, easy to use, and provides users with modularity depending on their need as determined by the insert geometry chosen to work with the cassette system.

The cassette-insert system of the present invention is unique. It allows for cell culture to take place outside of the confine of a small microfluidic channel which is challenging to culture in due to high shear stresses and low media and/or nutrient volumes. The insert may be put into the cassette, but may also be non-destructively removed from the cassette to facilitate end point assays. Polydimethylsiloxane (PDMS), a common material used in microfluidic organ on a chip systems, may be omitted from the present system.

The insert is modular so that it may be designed to be multiple geometries. One iteration is an insert with a polycarbonate membrane as the cell support or scaffold. Another iteration is an insert with a through hole for the user to add a natural or synthetic vitrified membrane or hydrated gel to the system. A hydrated gel may be added without any other support (e.g., a polycarbonate membrane or guide posts).

The inventors have advanced the technology of the present invention by developing an insert which allows for widespread use of organ-on-chip technologies with greater physiological cellular organization and biophysical forces (e.g., fluid flow and ECM stiffness). This device and insert design allow for easy addition of thin, vitrified ECM membranes. These membranes are both spatially and geometrically controllable through simple pipetting. The inventors have made thin membranes from collagen-I, a mixture of collagen and fibronectin, human tissue derived ECM, and/or Matrigel. Similarly, thick hydrated ECM gels may also be incorporated into the insert and device. The inventors have successfully incorporated hydrated gels such as collagen-I gel and decellularized placental ECM gel into a microfluidic device according to the present invention.

The term “cultured cells” as used herein refers to cells grown outside their natural environment. For example, the cultured cells may be cells that have been seeded onto an insert and grown in a culture medium on the insert. The cultured cells may be in a monolayer. The cultured cells may be in a monolayer attached to a surface of a vitrified membrane, a hydrated gel, or a semi-permeable membrane. The cultured cells may be in a hydrated gel. The cultured cells may be epithelial cells, stromal cells, endothelial cells, or a combination thereof.

The term “extracellular matrix (ECM)” as used herein refers to a network of proteins and other molecules that surround and support cells in a mammalian tissue.

The term “extracellular matrix (ECM) digest” as used herein refers to a mixture of a mammalian tissue and a digestion enzyme (e.g., pepsin).

The term “extracellular matrix (ECM) protein” as used herein refers to a protein that is found in an ECM.

The term “decellularized placental extracellular matrix (ECM)” as used herein refers to a mixture of ECM proteins isolated from a human placenta. The decellularized placental ECM may be prepared by removing the cellular matter from a human placenta with alcohol washes or hypertonic washes and then digesting the remaining tissue with pepsin and lyophilizing the digestion solution to generate lyophilized proteins. The lyophilized proteins may then be dissolved into acetic acid and gelled via neutralization with NaCI and salt balanced with PBS to produce a decellularized placental ECM solution. A ECM solution may also be made by mixing ECM proteins isolated from multiple sources to create a mixture of the proteins at, for example, predetermined concentrations. The ECM proteins used to create the hydrated gel or the vitrified membrane or coat the semi-permeable membrane may comprise collagens (e.g., collagen-I, collagen-IV), elastins, fibronectins, laminins, Matrigel, decellularized organ ECM or a combination thereof. For example, the ECM may be a mixture of collagen and fibronectin. A decellularized ECM may also prepared similarly as a mixture of ECM proteins isolated from another mammalian tissue.

The term “vitrified membrane” as used herein refers to a dried or dehydrated protein solution in the form of a thin layer. The protein solution may comprise an ECM digest, an individual protein naturally found in a tissue ECM (also known as ECM protein), or a mixture of ECM proteins.

The terms “hydrated gel” and “hydrogel” are used herein interchangeably and refer to a solid three-dimensional network having a liquid within the network. The hydrogel may comprise an ECM digest, an individual ECM protein, a mixture of ECM proteins. The hydrogel may comprise a synthetic hydrogel scaffold decorated with an ECM protein or a peptide comprising a functional binding domain of an ECM protein.

The term “semi-permeable membrane” as used herein refers to a plastic or polymer membrane with pores for solute and solvent diffusion or convection.

The term “epithelial cells” as used herein refers to a cell type that is isolated from an epithelial tissue or generated from a stem cell into an epithelial cell lineage.

The epithelial cells may be BeWo b30 cells, A2EN cells, VK2 cells, or Madin-Darby canine kidney (MDCK). The A 2EN cells may be present 2×10⁵ cells/insert after a 30-minute adherence time. The VK2 cells may be present at 2×10⁵ cells/insert after a 30-minute adherence time. The MDCK may be present at 160,000 cells/insert after a 4-hour adherence time.

The term “stromal cells” as used herein refers to a cell type that is isolated from the tissue stroma or generated from a stem cell into a mesenchymal or lymphocyte lineage. The stromal cells may be fibroblasts such as fibroblasts, innate immune cells, recruited immune cells, nerve cells, and smooth muscle cells. The fibroblasts may be present at 2×10⁵ cells/insert after a 30-minute adherence time.

The term “endothelial cells” as used herein refers to a cell type that is isolated from lymphatic or blood vasculature. The endothelial cells may be human umbilical vein endothelial cells (HUVECs) or human placental vascular endothelial cells (HPVECs). The HUVECs may be present at 150,000 cells/insert after a 4-hour adherence time.

The term “isolated or reconstructed vaginal microbial communities” as used herein refers to a population of microbiota that where each of the species have been isolated from patients and combined together based on population level abundance data or lab species that are combined together based on population level abundance data. The isolated or reconstructed vaginal microbial communities may be on top of epithelial cells.

The term “cassette” as used herein refers to an assembly of a base and a lid that may be used to clamp around an insert to create a sealed system having one or more fluid channels and connection points to perfuse the fluid channels in the system. The term “insert” as used herein refers to a structure that houses a cell culture platform and is compressible to create a sealed system when clamped into a cassette. The insert may comprise multiple pieces of compressible material adhered together with a commercial double sided adhesive film.

The term “cell culture platform” as used herein refers to a part of an insert on which cells are cultured.

The term “channel” as used herein refers to a space through which a fluid flows when an insert is clamped into a cassette.

The present invention provides a system for culturing cells. The system comprises a cassette, an insert in the cassette, and first cultured cells. The cassette comprises a lid and a base. The lid comprises a top channel inlet, a top channel outlet, a bottom channel inlet and a bottom channel outlet. The top channel inlet, the top channel outlet, the bottom channel inlet and/or bottom channel outlet may be alternatively in the base. The insert is between the lid and the base. The insert comprises a top layer having a top channel, a bottom layer having a bottom channel, and a cell culture platform. The insert may further comprise a third layer. The cell culture platform has a top platform surface in contact with the top channel and a bottom platform surface in contact with the bottom channel. The cell culture platform may be selected from the group consisting of a vitrified membrane, a hydrated gel, a semi-permeable membrane and a combination thereof. The top channel is sealed by the lid and the top platform surface and connected with the top channel inlet and the top channel outlet. A top flowing fluid moves to create a wall shear stress in the top channel through the top channel inlet, the top channel, and the top channel outlet. The bottom channel is sealed by the base and the bottom platform surface, and connected with the bottom channel inlet and the bottom channel outlet. The bottom flowing fluid moves to create a wall shear stress in the bottom channel through the bottom channel inlet, the bottom channel, and the bottom channel outlet.

The wall shear stress in the top channel may be about 0.01-1,000, 0.01-500, 0.01-100, 0.01-75, 0.01-50, 0.01-10, 0.01-1, 0.01-0.1, 0.1-1,000, 0.1-500, 0.1-100, 0.1-75, 0.1-50, 0.1-10, 0.1-1, 0.25-1,000, 0.25-500, 0.25-100, 0.25-75, 0.25-50, 0.25-10, 0.25-1, 0.5-1,000, 0.5-500, 0.5-100, 0.5-75, 0.5-50, 0.5-10, 0.5-1, 1-1,000, 1-500, 1-100, 1-75, 1-50 or 1-10 dyne/cm².

The wall shear stress in the bottom channel may be about 0.01-1,000, 0.01-500, 0.01-100, 0.01-75, 0.01-50, 0.01-10, 0.01-1, 0.01-0.1, 0.1-1,000, 0.1-500, 0.1-100, 0.1-75, 0.1-50, 0.1-10, 0.1-1, 0.25-1,000, 0.25-500, 0.25-100, 0.25-75, 0.25-50, 0.25-10, 0.25-1, 0.5-1,000, 0.5-500, 0.5-100, 0.5-75, 0.5-50, 0.5-10, 0.5-1, 1-1,000, 1-500, 1-100, 1-75, 1-50 or 1-10 dyne/cm².

The first cultured cells may be attached to the top platform surface and exposed directly to the top flowing fluid. The first cultured cells may be in a monolayer attached to the top platform surface and exposed directly to the top flowing fluid.

The first cultured cells may be attached to the bottom platform surface and exposed directly to the bottom flowing fluid. The first cultured cells may be in a monolayer attached to the bottom platform surface and exposed directly to the bottom flowing fluid.

The first cultured cells may be in the cell culture platform and exposed indirectly to the top flowing fluid and indirectly to the bottom flowing fluid.

The first cultured cells may be epithelial cells, stromal cells, or endothelial cells. The first cultured cells may be derived from a human tissue.

The vitrified membrane may comprise first ECM proteins. The first cultured cells may be attached to a surface of the vitrified membrane.

The hydrated gel may comprise second ECM proteins. The first cultured cells may be in the hydrated gel.

The hydrated gel may be prepared by technically known in the art. The hydrogel may comprise collagen-I (also referred to as a collagen-I gel) or decellularized placental ECM (also referred to as a decellularized placental ECM gel).

The collagen-I gel may be a hydrogel that supports cell growth in and on the gel made up entirely of the protein collagen-I in acetic acid and phosphate buffered saline (PBS) to control the concentration. gelation may be initiated by neutralizing the solution with NaCl.

The decellularized placental ECM gel may be a hydrogel that supports cell growth in and on the gel made up of ECM proteins that have been processed and extracted from human placental tissue and dissolved in acetic acid. The decellularized placental ECM gel comprises a wide variety of proteins, but not cells. A solution of decellularized placental ECM may comprise PBS to control the concentration and/or may be neutralized with NaCl to initiate gelation.

The semi-permeable membrane may be a polycarbonate membrane or a polyethylene terephthalate (PET) membrane.

The semi-permeable membrane may have a semi-permeable membrane surface coated with third ECM proteins. The first cultured cells may be attached to the coated semi-permeable membrane surface.

The vitrified membrane may have a thickness of about 0.1-50, 0.1-10, 0.1-1, 1-50, 1-10, 2-1,000, 2-50, 2-10, 5-50, 5-10, or 10-50 μm. The vitrified membrane may comprise collagen-I, fibronectin or Matrigel.

The culture platform may consist of the vitrified membrane. The vitrified membrane may have a top vitrified membrane surface and a bottom vitrified membrane surface. The top vitrified membrane surface may be the top platform surface. The bottom vitrified membrane surface may be the bottom platform surface. The first cultured cells may be attached to the top or bottom vitrified membrane surface. The first cultured cells may be in a monolayer attached to the top or bottom vitrified membrane surface.

Where the culture platform consists of a vitrified membrane having a top vitrified membrane surface and a bottom vitrified membrane surface, the system may further comprise second cultured cells. The first cultured cells and the second cultured cells may be the same or different. The second cultured cells may be epithelial cells, stromal cells, or endothelial cells. The system may further comprise isolated or reconstructed vaginal microbial communities. The isolated or reconstructed vaginal microbial communities may be on top of the epithelial cells.

Where the culture platform consists of a vitrified membrane having a top vitrified membrane surface and a bottom vitrified membrane surface and the system comprises first cultured cells and second cultured cells, each of the first cultured cells and the second cultured cells may be attached to the top vitrified membrane surface or the bottom vitrified membrane surface. The first cultured cells may be attached to the top vitrified membrane surface and exposed directly to the top flowing fluid while the second cultured cells may be attached to the bottom vitrified membrane surface and exposed directly to the bottom flowing fluid. The first cultured cells may be attached to the bottom vitrified membrane surface and exposed directly to the bottom flowing fluid and the second cultured cells may be attached to the top vitrified membrane surface and exposed directly to the top flowing fluid. The first cultured cells may be in a monolayer. The second cultured cells may be in a monolayer. Each of the first cultured cells and the second cultured cells may be in a monolayer.

Where the culture platform consists of a vitrified membrane having a top vitrified membrane surface and a bottom vitrified membrane surface, the system may comprise epithelial cells and stromal cells. The vitrified membrane may comprise collagen-I. The epithelial cells may be in a monolayer attached to the top vitrified membrane surface and exposed directly to the top flowing fluid while the stromal cells may be in a monolayer attached to the bottom vitrified membrane surface and exposed directly to the bottom flowing fluid. The epithelial cells may be in a monolayer attached to the bottom vitrified membrane surface and exposed directly to the bottom flowing fluid while the stromal cells may be in a monolayer attached to the top vitrified membrane surface and exposed directly to the top flowing fluid. The system may further comprise isolated or reconstructed vaginal microbial communities. The isolated or reconstructed vaginal microbial communities may be on top of the epithelial cells.

Where the culture platform consists of a vitrified membrane having a top vitrified membrane surface and a bottom vitrified membrane surface, the system may comprise epithelial cells and endothelial cells. The vitrified membrane may comprise collagen-I. The epithelial cells may be in a monolayer attached to the top vitrified membrane surface and exposed directly to the top flowing fluid while the endothelial cells may be in a monolayer attached to the bottom vitrified membrane surface and exposed directly to the bottom flowing fluid. The epithelial cells may be in a monolayer attached to the bottom vitrified membrane surface and exposed directly to the bottom flowing fluid while the endothelial cells may be in a monolayer attached to the top vitrified membrane surface and exposed directly to the top flowing fluid.

Where the culture platform consists of a vitrified membrane having a top vitrified membrane surface and a bottom vitrified membrane surface and the vitrified membrane comprises collagen-I, the system may comprise epithelial cells and endothelial cells. The epithelial cells may be attached to the top vitrified membrane surface while the endothelial cells are attached the bottom vitrified membrane surface. The epithelial cells may be attached to the bottom vitrified membrane surface while the endothelial cells are attached the top vitrified membrane surface. The epithelial cells may be BeWo b30 cells, A2EN cells, VK2 cells, or Madin-Darby canine kidney (MDCK). The endothelial cells may be human umbilical vein endothelial cells (HUVECs) or human placental vascular endothelial cells (HPVECs). For example, the culture platform may consist of the vitrified membrane, the vitrified membrane comprises collagen-I, the epithelial cells are BeWo b30 cells and the endothelial cells are HUVECs.

The hydrated gel may have a thickness of about 50-5,000, 50-4,500, 50-4,000,50-3,500, 50-2,500, 50-2,000, 50-1500, 50-1,000, 50-500 or 50-100 μm. The hydrated gel may comprise collagen-I or decellularized placental extracellular matrix (ECM).

The cell culture platform may consist of the hydrated gel. The hydrated gel may have a top gel surface and a bottom gel surface. The top gel surface may be the top platform surface. The bottom gel surface may be the bottom platform surface. The first cultured cells may be in the hydrated gel. The first cultured cells may be attached to the top or bottom gel surface. The first cultured cells may be in a monolayer attached to the top or bottom gel surface.

Where the cell culture platform consists of a hydrated gel having a top gel surface and a bottom gel surface, the system may further comprise second cultured cells. The first cultured cells and the second cultured cells may be the same or different. The second cultured cells may epithelial cells, stromal cells, or endothelial cells. The system may further comprise isolated or reconstructed vaginal microbial communities. The isolated or reconstructed vaginal microbial communities may be on top of the epithelial cells.

Where the cell culture platform consists of a hydrated gel having a top gel surface and a bottom gel surface and the system comprises first cultured cells and second cultured cells, each of the first cultured cells and the second cultured cells may be in the hydrated gel or attached to the top or bottom gel surface. The first cultured cells may be in the gel while the second cultured cells may be attached to the top gel surface and exposed directly to the top flowing fluid. The first cultured cells may be in the gel while the second cultured cells may be attached to the bottom gel surface and exposed directly to the bottom flowing fluid. The first cultured cells may be attached to the top gel surface and exposed to the top flowing fluid while the second cultured cells may be attached to the bottom gel surface and exposed directly to the bottom flowing fluid. The first cultured cells may be attached to the bottom gel surface and exposed to the bottom flowing fluid while the second cultured cells may be attached to the top gel surface and exposed directly to the top flowing fluid.

Where the culture platform consists of a hydrated gel having a top gel surface and a bottom gel surface, the system may comprise epithelial cells and stromal cells. The epithelial cells may be attached to the top gel surface while the stromal cells may be in the hydrated gel. The epithelial cells may be attached to the bottom gel surface while the stromal cells may be in the hydrated gel. The epithelial cells may be attached to the top gel surface while the stromal cells may be attached to the bottom gel surface. The epithelial cells may be attached to the bottom gel surface while the stromal cells may be attached to the top gel surface.

Where the culture platform consists of a hydrated gel having a top gel surface and a bottom gel surface, the system may comprise epithelial cells, stromal cells and isolated or reconstructed vaginal microbial communities. The epithelial cells may be attached to the top gel surface, the stromal cells may be in the hydrated gel, and the isolated or reconstructed vaginal microbial communities may be on the epithelial cells. The epithelial cells may be attached to the bottom gel surface, the stromal cells may be in the hydrated gel and the isolated or reconstructed vaginal microbial communities may be on the epithelial cells. The epithelial cells may be attached to the top gel surface, the stromal cells may be attached to the bottom gel surface, and the isolated or reconstructed vaginal microbial communities may be on the epithelial cells. The epithelial cells may be attached to the bottom gel surface, the stromal cells may be attached to the top gel surface, and the isolated or reconstructed vaginal microbial communities may be on the epithelial cells.

Where the culture platform consists of a hydrated gel having a top gel surface and a bottom gel surface, the system may comprise epithelial cells and endothelial cells. The epithelial cells may be attached to the top gel surface while the endothelial cells may be in the hydrated gel. The epithelial cells may be attached to the bottom gel surface while the endothelial cells may be in the hydrated gel. The epithelial cells may be attached to the top gel surface while the endothelial cells may be attached to the bottom gel surface. The epithelial cells may be attached to the bottom gel surface while the endothelial cells may be attached to the top gel surface. The system may further comprise isolated or reconstructed vaginal microbial communities. The isolated or reconstructed vaginal microbial communities may be on top of the epithelial cells.

Where the cell culture platform consists of a hydrated gel having a top gel surface and a bottom gel surface and the system comprises first cultured cells, second cultured cells and third cultured cells, each of the first cultured cells, the second cultured cells, and the third cultured cells may be in the hydrated gel or attached to the top or bottom gel surface. The first cultured cells may be in the gel, the second cultured cells may be attached to the top gel surface and exposed directly to the top flowing fluid, and the third cultured cells may be attached to the bottom gel surface and exposed directly to the bottom flowing fluid.

Where the cell culture platform consists of a hydrated gel having a top gel surface and a bottom gel surface, the system may comprise epithelial cells, stromal cells, and endothelial cells. The epithelial cells may be attached to the top gel surface, the stromal cells may be in the hydrated gel, and the endothelial cells may be attached to the bottom gel surface. The epithelial cells may be attached to the bottom gel surface, the stromal cells may be in the hydrated gel, and the endothelial cells may be attached to the top gel surface.

Where the cell culture platform consists of a hydrated gel having a top gel surface and a bottom gel surface, the system may comprise epithelial cells, stromal cells, endothelial cells, and isolated or reconstructed vaginal microbial communities. The epithelial cells may be attached to the top gel surface, the stromal cells may be in the hydrated gel, the endothelial cells may be attached to the bottom gel surface, and the isolated or reconstructed vaginal microbial communities may be on top of the epithelial cells. The epithelial cells may be attached to the bottom gel surface, the stromal cells may be in the hydrated gel, the endothelial cells may be attached to the top gel surface, and the isolated or reconstructed vaginal microbial communities may be on top of the epithelial cells.

Where the culture platform consists of the hydrated gel and the hydrated gel comprises collagen-I or decellularized placental extracellular matrix (ECM) and has a top gel surface as the top platform surface and a bottom gel surface as the bottom platform surface, the system may comprise stromal cells, epithelial cells, and endothelial cells. The stromal cells may be in the hydrated gel, the epithelial cells may be attached to the top gel surface, and the endothelial cells may be attached to the bottom gel surface. The stromal cells may be in the hydrated gel, the epithelial cells may be attached to the bottom gel surface, and the endothelial cells may be attached to the top gel surface. The stromal cells may be BJ fibroblasts. The epithelial cells may be BeWo cells or A2EN cells. The endothelial cells may be Human umbilical vein endothelial cells (HUVECs) or human placental vascular endothelial cells (HPVECs). In one embodiment, the first cultured cells are BJ fibroblasts, for example, in the hydrated gel, the second cultured cells are BeWo cells, for example, attached to the top gel surface, and the third cultured cells are Human umbilical vein endothelial cells (HUVECs), for example, attached to the bottom gel surface.

The cell culture platform may consist of the semi-permeable membrane. The semi-permeable membrane may have a first semi-permeable membrane surface and a second semi-permeable membrane surface. The first semi-permeable membrane surface may be the top platform surface while the second semi-permeable membrane surface may be the bottom platform surface. The first semi-permeable membrane surface may be the bottom platform surface while the second semi-permeable membrane surface may be the top platform surface. The first cultured cells may be attached to the first or second semi-permeable membrane surface. The first or second semi-permeable membrane surface may be coated with ECM proteins. The first cultured cells may be attached to the coated first or second semi-permeable membrane surface. The system may further comprise second cultured cells.

Where the culture platform consists of a semi-permeable membrane having a first semi-permeable membrane surface as the top platform surface, which is also referred to as a top semi-permeable membrane surface, and a second semi-permeable membrane surface as the bottom platform surface, which also referred to as a bottom semi-permeable membrane surface, the system may further comprise second cultured cells. The first cultured cells and the second cultured cells may be the same or different. The second cultured cells may epithelial cells, stromal cells, or endothelial cells. The system may further comprise isolated or reconstructed vaginal microbial communities. The isolated or reconstructed vaginal microbial communities may be on top of the epithelial cells. Each of the first cultured cells and the second cultured cells may be attached to the top or bottom semi-permeable membrane surface. The top semi-permeable membrane surface may be coated with ECM proteins. The bottom semi-permeable membrane surface may be coated with ECM proteins. The top semi-permeable membrane surface and bottom semi-permeable membrane surface may be coated with the same ECM proteins. The first cultured cells may be attached to the top semi-permeable membrane surface and exposed directly to the top flowing fluid while the second cultured cells may be attached to the bottom semi-permeable membrane surface and exposed directly to the bottom flowing fluid. The first cultured cells may be attached to the bottom semi-permeable membrane surface and exposed directly to the bottom flowing fluid and the second cultured cells may be attached to the top semi-permeable membrane surface and exposed directly to the top flowing fluid. The first cultured cells may be in a monolayer. The second cultured cells may be in a monolayer. Each of the first cultured cells and the second cultured cells may be in a monolayer.

Where the culture platform consists of a semi-permeable membrane having a top semi-permeable membrane surface and a bottom semi-permeable membrane surface, the system may comprise epithelial cells and stromal cells. The epithelial cells may be attached to the top semi-permeable membrane surface while the stromal cells may be attached to the bottom semi-permeable membrane surface. The epithelial cells may be attached to the bottom semi-permeable membrane surface while the stromal cells may be attached to the top semi-permeable membrane surface.

Where the culture platform consists of a semi-permeable membrane having a top semi-permeable membrane surface and a bottom semi-permeable membrane surface, the system may comprise epithelial cells, stromal cells, and isolated or reconstructed vaginal microbial communities. The epithelial cells may be attached to the top semi-permeable membrane surface, the stromal cells may be attached to the bottom semi-permeable membrane surface and the isolated or reconstructed vaginal microbial communities may be on top of the epithelial cells. The epithelial cells may be attached to the bottom semi-permeable membrane surface, the stromal cells may be attached to the top semi-permeable membrane surface, and the isolated or reconstructed vaginal microbial communities may be on top of the epithelial cells.

Where the culture platform consists of a semi-permeable membrane having a top semi-permeable membrane surface and a bottom semi-permeable membrane surface, the system may comprise epithelial cells and endothelial cells. The epithelial cells may be attached to the top semi-permeable membrane surface while the endothelial cells may be attached to the bottom semi-permeable membrane surface. The epithelial cells may be attached to the bottom semi-permeable membrane surface while the endothelial cells may be attached to the top semi-permeable membrane surface.

Where the culture platform consists of a semi-permeable membrane having a top semi-permeable membrane surface and a bottom semi-permeable membrane surface, the system may comprise epithelial cells, endothelial cells, and isolated or reconstructed vaginal microbial communities. The epithelial cells may be attached to the top semi-permeable membrane surface, the endothelia cells may be attached to the bottom semi-permeable membrane surface and the isolated or reconstructed vaginal microbial communities may be on top of the epithelial cells. The epithelial cells may be attached to the bottom semi-permeable membrane surface, the endothelial cells may be attached to the top semi-permeable membrane surface, and the isolated or reconstructed vaginal microbial communities may be on top of the epithelial cells.

The culture platform may consist of the hydrated gel and the semi-permeable membrane. The hydrated gel may have a first gel surface and a second gel surface. The semi-permeable membrane may have a first semi-permeable membrane surface and a second semi-permeable membrane surface. The second gel surface and the first semi-permeable membrane surface may be in direct contact. The first gel surface may be the top platform surface while the second semi-permeable membrane surface may be the bottom platform surface. The first gel surface may be the bottom platform surface while the second semi-permeable membrane surface may be the top platform surface. The first cultured cells may be in the hydrated gel, attached to the first gel surface, or attached to the second semi-permeable membrane surface. The second semi-permeable membrane surface may be coated with ECM proteins. The system may further comprise second cultured cells. The system may further comprise second cultured cells and third cultured cells.

Where the culture platform consists of a hydrated gel having a first gel surface as the top platform surface and a second gel surface and a semi-permeable membrane having a first semi-permeable membrane surface in direct contact with the second gel surface and a second semi-permeable membrane surface as the bottom platform surface, the system may comprise first cultured cells and second cultured cells. Each of the first cultured cells and the second cultured cells may be in the hydrated gel or attached to the first gel surface or the second semi-permeable membrane surface. The first cultured cells may be attached to the first gel surface and the second cultured cells may be in the hydrated gel. The first cultured cells may be attached to the first gel surface, and the second cultured cells may be attached to the second semi-permeable membrane surface. The first cultured cells may be in the hydrated gel, and the second cultured cells may be attached to the second semi-permeable membrane surface. The second semi-permeable membrane surface may be coated with ECM proteins. The first gel surface may be the top platform surface while the second semi-permeable membrane surface may be the bottom platform surface. The first gel surface may be the bottom platform surface while the second semi-permeable membrane surface may be the top platform surface.

Where the culture platform consists of a hydrated gel having a first gel surface as the top platform surface and a second gel surface and a semi-permeable membrane having a first semi-permeable membrane surface in direct contact with the second gel surface and a second semi-permeable membrane surface as the bottom platform surface, the system may comprise first cultured cells, second cultured cells and third cultured cells. Each of the first cultured cells, the second cultured cells and third cultured cells may be in the hydrated gel or attached to the first gel surface or the second semi-permeable membrane surface. The first cultured cells may be attached to the first gel surface, the second cultured cells may be in the hydrated gel, and the third cultured cells may be attached to the second semi-permeable membrane surface. The second semi-permeable membrane surface may be coated with ECM proteins. The first gel surface may be the top platform surface while the second semi-permeable membrane surface may be the bottom platform surface. The first gel surface may be the bottom platform surface while the second semi-permeable membrane surface may be the top platform surface.

Where the culture platform consists of a hydrated gel having a first gel surface as the top platform surface and a second gel surface and a semi-permeable membrane having a first semi-permeable membrane surface in direct contact with the second gel surface and a second semi-permeable membrane surface as the bottom platform surface, the system may comprise epithelial cells, stromal cells and endothelial cells. The epithelial cells may be attached to the first gel surface, the stromal cells may be in the hydrated gel, and the endothelial cells may be attached to the second semi-permeable membrane surface. The second semi-permeable membrane surface may be coated with ECM proteins.

Where the culture platform consists of a hydrated gel having a first gel surface as the top platform surface and a second gel surface and a semi-permeable membrane having a first semi-permeable membrane surface in direct contact with the second gel surface and a second semi-permeable membrane surface as the bottom platform surface, the system may comprise epithelial cells, stromal cells, endothelial cells, and isolated or reconstructed vaginal microbial communities. The epithelial cells may be attached to the first gel surface, the stromal cells may be in the hydrated gel, the endothelial cells may be attached to the second semi-permeable membrane surface, and the isolated or reconstructed vaginal microbial communities may be on top of the epithelial cells. The second semi-permeable membrane surface may be coated with ECM proteins.

Where the culture platform consists of a hydrated gel having a first gel surface as the bottom platform surface and a second gel surface and a semi-permeable membrane having a first semi-permeable membrane surface in direct contact with the second gel surface and a second semi-permeable membrane surface as the top platform surface, the system may comprise epithelial cells, stromal cells and endothelial cells. The epithelial cells may be attached to the first gel surface, the stromal cells may be in the hydrated gel, and the endothelial cells may be attached to the second semi-permeable membrane surface. The second semi-permeable membrane surface may be coated with ECM proteins.

Where the culture platform consists of a hydrated gel having a first gel surface as the bottom platform surface and a second gel surface and a semi-permeable membrane having a first semi-permeable membrane surface in direct contact with the second gel surface and a second semi-permeable membrane surface as the top platform surface, the system may comprise epithelial cells, stromal cells, endothelial cells, and isolated or reconstructed vaginal microbial communities. The epithelial cells may be attached to the first gel surface, the stromal cells may be in the hydrated gel, the endothelial cells may be attached to the second semi-permeable membrane surface, and the isolated or reconstructed vaginal microbial communities may be on top of the epithelial cells. The second semi-permeable membrane surface may be coated with ECM proteins.

The insert may further comprise an adhesive film. Where the cell culture platform consists of the semi-permeable membrane. The semi-permeable membrane may be a polycarbonate membrane or a polyethylene terephthalate (PET) membrane. The insert may comprise two adhesive films, one between the top layer and the top platform surface and another one between the bottom layer and the cell culture platform surface. Where the cell culture platform consists of a vitrified membrane or a hydrated gel, the insert may comprise only one adhesive film between the top layer and the bottom layer.

The lid may further comprise a glass surface. The glass surface may be of a coverslip.

The top channel inlet may be connected to a first perfusion pump. The bottom channel inlet may be connected to a second perfusion pump. The first perfusion pump and the second perfusion pump may be the same.

The system may comprise polydimethylsiloxane (PDMS). The system may exclude polydimethylsiloxane (PDMS).

The top flowing fluid may comprise a top agent selected from the group consisting of media, cells, blood, microbiota, pharmacological agents, normoxic air, anoxic air and a combination. The top agent may migrate from the top channel into the cell culture platform. The top agent may migrate from the top fluidic channel through the cell culture platform and into the bottom channel.

The bottom flowing fluid may comprise a bottom agent selected from the group consisting of media, cells, blood, microbiota, pharmacological agents, normoxic air, anoxic air and a combination. The bottom agent may migrate from the bottom channel into the cell culture platform. The bottom agent may migrate from the bottom fluidic channel through the cell culture platform and into the top channel.

For each of the systems of the present invention, a method for growing cells is provided. The method comprises incubating the first cultured cells in the system. The first cultured cells may be in a monolayer attached to the top or bottom platform surface. The method may further comprise seeding cells onto the top platform surface or the bottom platform surface in the insert outside of the cassette such that the seeded cells are attached to the top or bottom platform surface. The method may further comprise growing the attached seeded cells to form a monolayer on the top or bottom platform surface. The insert having the first cultured cells in a monolayer attached to the top or bottom platform surface may be prepared after the attached seeded cells are grown for about 1-4 or 1-6 days. The method may further comprise placing the insert in the cassette to establish fluid flow. The method may further comprise removing the insert from the cassette. At least about 50%, 60%, 70%, 80%, 90%, 95%, 99% or 100% of the first cultured cells remain attached to the top or bottom platform surface after the removal. The method may further comprise characterizing the first cultured cells remaining attached to the cell culture platform in the insert after the removal.

The present invention also provides a method for preparing a system for culturing cells. The preparation method comprises fabricating a cassette with a lid and a base. The lid comprises a top channel inlet, a top channel outlet, a bottom channel inlet and a bottom channel outlet. The preparation method also comprises providing a cell culture platform having a top platform surface and a bottom platform surface. The cell culture platform is selected from the group consisting of a vitrified membrane, a hydrated gel, a semi-permeable membrane, and a combination thereof. The preparation method further comprises assembling an insert comprising the cell culture platform, a top layer having a top channel in contact with the top platform surface, and a bottom layer having a bottom channel in contact with the bottom platform surface; placing the insert in the cassette. The insert is between the lid and the base. The top channel is sealed by the lid and the top platform surface and connected with the top channel inlet and the top channel outlet, and the bottom channel is sealed by the base and the bottom platform and connected with the bottom channel inlet and the bottom channel outlet. The preparation method further comprises perfusing a top flowing fluid at a top channel shear stress through the top channel inlet, the top channel, and the top channel outlet, and perfusing a bottom flowing fluid at a bottom channel shear stress through the bottom channel inlet, the bottom channel, and the bottom channel outlet.

According to the preparation method, the wall shear stress in the top channel may be about 0.01-1,000, 0.01-500, 0.01-100, 0.01-75, 0.01-50, 0.01-10, 0.01-1, 0.01-0.1, 0.1-1,000, 0.1-500, 0.1-100, 0.1-75, 0.1-50, 0.1-10, 0.1-1, 0.25-1,000, 0.25-500, 0.25-100, 0.25-75, 0.25-50, 0.25-10, 0.25-1, 0.5-1,000, 0.5-500, 0.5-100, 0.5-75, 0.5-50, 0.5-10, 0.5-1, 1-1,000, 1-500, 1-100, 1-75, 1-50 or 1-10 dyne/cm².

According to the preparation method, the wall shear stress in the top channel may be about 0.01-1,000, 0.01-500, 0.01-100, 0.01-75, 0.01-50, 0.01-10, 0.01-1, 0.01-0.1, 0.1-1,000, 0.1-500, 0.1-100, 0.1-75, 0.1-50, 0.1-10, 0.1-1, 0.25-1,000, 0.25-500, 0.25-100, 0.25-75, 0.25-50, 0.25-10, 0.25-1, 0.5-1,000, 0.5-500, 0.5-100, 0.5-75, 0.5-50, 0.5-10, 0.5-1, 1-1,000, 1-500, 1-100, 1-75, 1-50 or 1-10 dyne/cm².

According to the preparation method, first cultured cells may be in a monolayer attached to the top platform surface and exposed directly to the top channel and the top flowing fluid. The first cultured cells may be in a monolayer attached to the bottom platform surface and exposed directly to the bottom channel and the bottom flowing fluid.

The preparation method may further comprise seeding cells onto the top platform surface or the bottom platform surface in the insert outside of the cassette such that the seeded cells are attached to the top or bottom platform surface; and growing the attached seeded cells to form a monolayer. The insert having the first cultured cells in a monolayer attached to the top or bottom platform surface is thereby prepared. In some embodiments, second cultured cells may be attached the other side of the platform surface in a similar way.

According to the preparation method, first cultured cells may be in the cell culture platform and exposed indirectly to the top flowing fluid and indirectly to the bottom flowing fluid. The insert may further comprise a third layer between the top layer and the bottom layer.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

### Example 1. Methods for Cassette Fabrication and Use

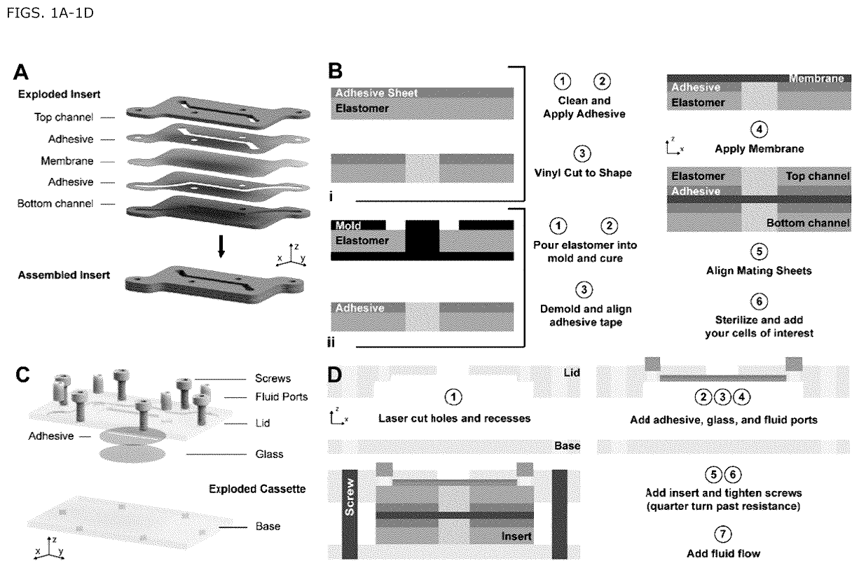

A cassette is fabricated for all different types of inserts. The cassette has two parts—a lid and a base. Acrylic sheets (McMaster-Carr, 8560K211) were laser cut using a 40 W CO₂ laser and a Laser Muse 1.5″ ZnSe Focus Lens (Full Spectrum Laser, Hobby Series 20×12). For the cassette base, vector cut holes were made to allow tapping of M2 screws. For the cassette lid, layers were rastered twice for an insert recess and a circular glass coverslip (No 1.5, ∅15 mm). Vector cut clearance holes allowed for M2 screw placement. In the raster layer containing the glass coverslip, the area was sanded flat or smoothed with heat to prevent coverslip breakage, and a vinyl cut adhesive sheet (3M 9500PC) was adhered. The circular coverslip was then adhered to the adhesive. (FIGS. 1C and 1D). The coverslip was flush against the other rastered surface so when the insert was placed against the acrylic lid it was also flush.

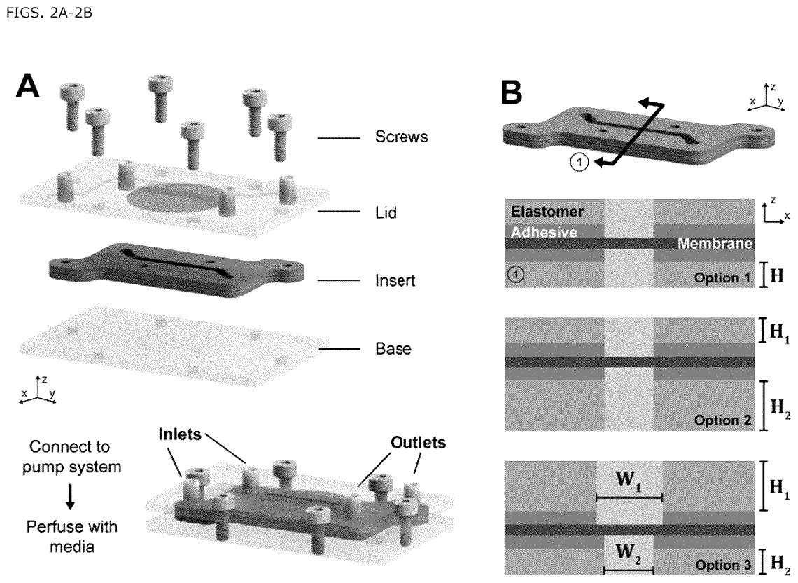

Any insert may be placed on the cassette base and the rastered lid may be placed on the insert aligning it. Then the lid may be screwed to the base, clamping the insert in place and aligned with the fluid ports (FIGS. 2A-2B).

### Example 2. Methods for Polycarbonate Membrane Laden Insert Fabrication

For inserts, double-sided adhesive film (3M 9500PC) was placed on one side of 1/32″ silicone sheets (McMaster-Carr, 86465K21) taking care not to create air pockets between the layers. Any large bubbles present were rolled out using a western blot roller (SNAP2RL, Millipore-Sigma), taking note not to press too firmly and stretch/curl the silicone sheet. With the paper backing on, the laminated silicone and adhesive were fed into a vinyl cutter (USCutter, SC or Titan3) silicone side up and the upper and lower insert patterns (made in SolidWorks converted to .SVG in Adobe Illustrator) were traced with a 45° blade at 100 mm/s and 300 g of pressure for SC or 12 mm/s and 280 g of pressure for Titan 3. In a laminar flow hood, each silicone insert was assembled using a press. The lower laminated channel was placed on the lower press platen. A 3 μm polycarbonate membrane (Cytiva, 10418306) was placed using forceps and a silicone stamp pressed it against the lower laminated channel to reduce wrinkling of the membrane. The upper laminated channel was placed onto the upper platen of the custom press. After placement of the polycarbonate membrane, the upper platen was pressed onto the lower platen, piercing through alignment holes in the lower platen and through the membrane (FIGS. 1A and 1Bi). The alignment holes were offset from the cell culture region on the membrane to prevent tearing.

The insert may also be made from commercially available polyurethane. For example, GSP-1552-2 (GSPolymers) was mixed 1:1 per the provided company protocol. The material was allowed to cure for 48 hours at room temp in an open-faced mold exposed to air in a fume hood. The cured polyurethane was demolded and similar techniques as described above in Example 1 used to apply the adhesive and the polycarbonate membrane.

### Example 3. Methods for Vitrified/Hydrated Gel Laden Insert Fabrication

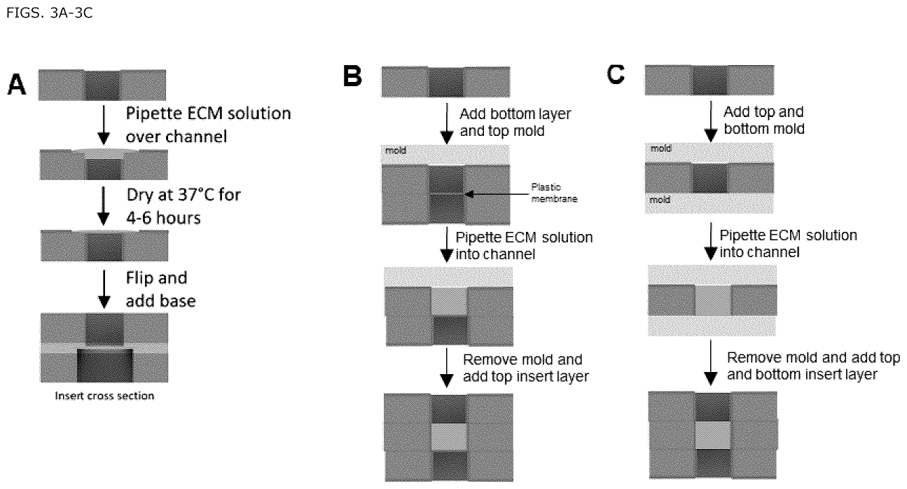

There are multiple different inserts created for addition of an ECM cell scaffold. (1) A similar 2-layer insert to the semi-permeable membrane inserts, sans the membrane, (2) an insert with 3 silicone layers with a semi-permeable membrane between the middle and bottom layers, and (3) a 3 layer insert without a semi-permeable membrane. To fabricate (1) the same methods described above in Example 2 are used except for the addition of the semi-permeable membrane (FIG. 3A). To fabricate (2) a top mold was added to a two layer insert with a semi-permeable membrane. The ECM solution was pipetted into the resulting channel and allowed to gel before the mold was removed and the top layer of the insert was adhered (FIG. 3B). To fabricate (3) a top and bottom mold were added to the middle layer of the insert. The ECM solution was pipetted into the resulting channel and allowed to gel before the molds were removed and the top and bottom layers of the insert were adhered (FIG. 3C).

In inserts with a hydrated gel, (3-Aminopropyl)triethoxysilane (APTES) and glutaraldehyde (GA) or polydopamine were used to functionalize the surface of the silicone insert channel in order to covalently bond the ECM to the material as previously described. For APTES/GA functionalization, the silicone inserts were plasma cleaned (Harrick Plasma, PDC-001-HP) on High intensity for 60 s and then quickly submerged in a 10% APTES solution in 95% anhydrous biotechnology grade ethanol (IBI Scientific) for 15 min at room temperature. The inserts were washed thoroughly with excess ethanol 3 times then in DI water 3 times to remove unreacted APTES then dried with nitrogen. Then, 2.5% GA in DI water was pipetted directly into the insert channel for 30 min. The insert was washed 3× in DI water and dried with nitrogen. Inserts were either used immediately or stored in DI water at 4° C. until use. For polydopamine functionalization, 2 mg/mL polydopamine in 10 nM Tris in distilled water was pipetted into the channel and incubated for 2 hours at room temperature. After 2 hours, the insert was washed 3× with distilled water and left to dry at room temperature or 37° C.

To create collagen-I vitrified membranes, collagen-I was isolated from rat tails as previously described. Lyophilized collagen-I was dissolved in 0.1% glacial acetic acid at a concentration of 10 mg/mL to create the pre-gel solution. To ensure sterility, inserts were washed with 70% ethanol before the remaining steps were completed in a biosafety cabinet. Pre-gel solution was diluted to 8 mg/mL and neutralized and salt balanced using 1N NaCl and 10× HBSS, respectively and 1× PBS was added until the final desired volume and concentration was reached. The insert was suspended by resting the edges on polymethylsiloxane (PDMS) supports and the neutralized collagen solution was quickly pipetted into the insert channel, allowed to gel, and then dry overnight at 37° C. Thin collagen membranes could be stored at 4° C. for up to a week before use. The same protocol could be used with collagen-I mixed with fibronectin, Matrigel, or decellularized ECM.

To create hydrated ECM stroma, the same procedure was followed except lyophilized collagen-I or decellularized placental ECM was dissolved at 8 mg/mL and diluted to a 5 mg/mL neutralized solution. Once pipetted into the APTES/GA functionalized insert, the gel was allowed to gel for 30 min at 37° C. Hydrated gels were used immediately after gelation.

### Example 4. Methods for Cell Culture on Insert With Semi-Permeable Membrane or Vitrified ECM Membrane

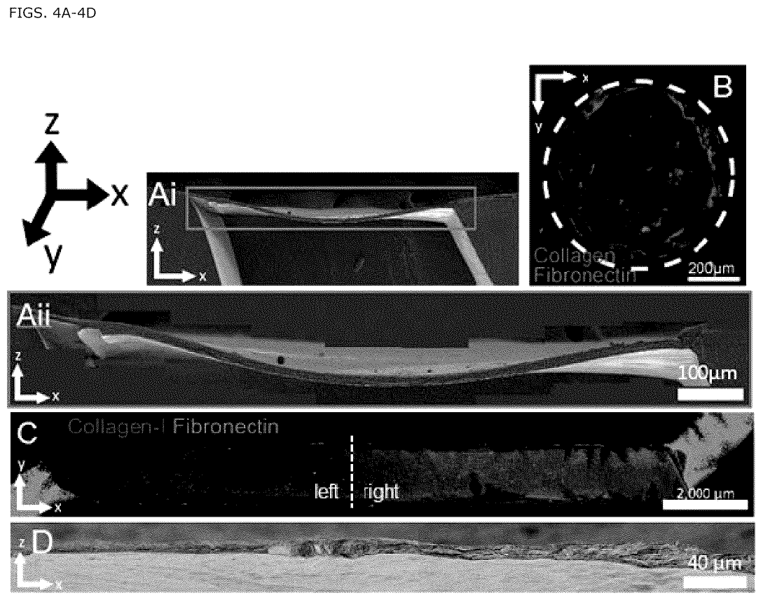

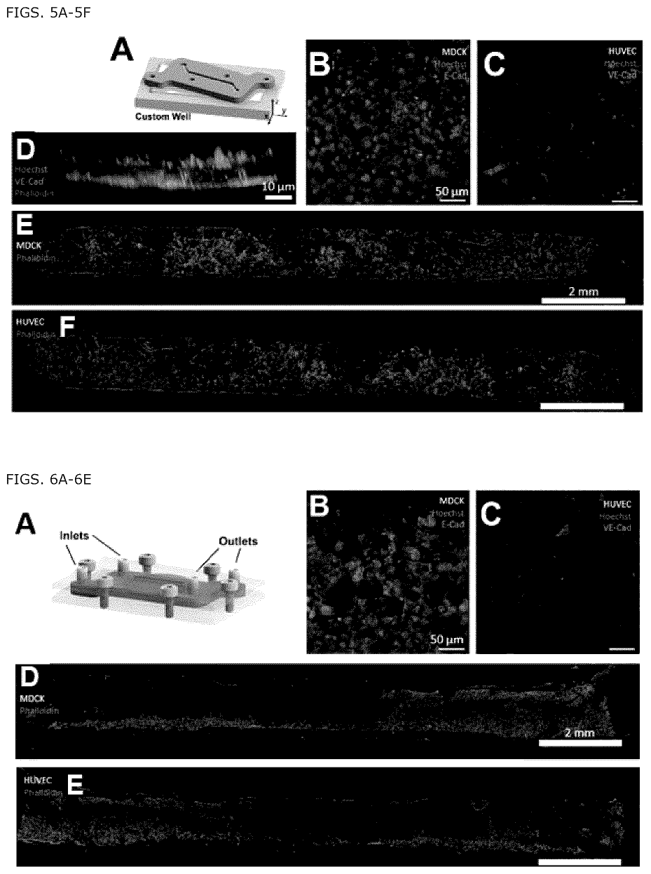

Techniques similar to those described in this example may be used for any type of cell system. Depending on the type of the cultured cells, the ECM protein coating on the semi-permeable membrane, the type of ECM used for the vitrified membrane, the number of the cells initially seeded, and the time in which monolayer formation occurs may vary. Examples of these variations for some tissues are detailed in Example 7-10. The polycarbonate membrane of the insert was coated with proteins (ex. 0.1% gelatin, 0.5 mg/mL fibronectin) for 45 minutes at 37° C. on both sides, after which the excess gelatin was removed. Endothelial cells were seeded on the top channel at a density of 150,000 cells/insert and allowed to adhere for 4 hours. After 4 hours, media was added to the PDMS well that the insert was rested on. Endothelial cells were cultured for 24 hours, after which the insert was flipped and epithelial cells were seeded on the bottom channel at a seeding density of 160,000 cells/insert. Inserts were statically cultured (FIG. 5A) until endothelial and epithelial monolayers formed, around 3 days, as confirmed by E-Cadherin, VE-Cadherin, and phalloidin immunofluorescent staining (FIG. 5B-F). For dynamic conditions, after monolayer formation, the insert was clamped into the cassette and flow was initiated (FIG. 6A).

Flow was maintained at 1.27 μL/min for the epithelial channel and progressively ramped up from 30 μL/min to 3 mL/min over the span of 48 hours for the endothelial channel to induce shear stress responses at physiologic ranges. Cell phenotype and monolayer formation confirmed by immunofluorescent staining (FIG. 6B-E).

### Example 5. Methods for Cell Culture on Insert Hydrated Gel or a Hydrated Gel on a Semi-Permeable Membrane

Techniques similar to those described in this example may be used for any type of cell system. Depending on the type of the cultured cells, the ECM protein coating on the semi-permeable membrane, the number of the cells initially seeded, and the time in which monolayer formation occurs may vary.

Epithelial cells, fibroblasts, and endothelial cells may be used. Fibroblasts were suspended in neutralized collagen-I solution at a density of 600,000 cells/mL. The solution was introduced into the insert using sterile conditions as described above in Example 3 and allowed to gel for at least 30 min. Endothelial cells were seeded on the hydrogel surface at a density of 50,000 cells/gel and allowed to adhere for 4 hrs. After 4 hrs the insert was flipped and the PDMS well support was filled with endothelial cell media (ScienCell-ECM media). The epithelial cells were then seeded on the other side of the gel at a density of 100,000 cells/insert in 10-20 μL of media and allowed to adhere for at least 1 hr before adding more media (ATCC—F-12K with 10% FBS, 1% penicillin/streptomycin). Visual confirmation of monolayers was done with select inserts via immunofluorescent staining with anti-E-cadherin, anti-VE-cadherin, and phalloidin and imaged on a confocal microscope (Zeiss LSM 800) (FIG. 7).

### Example 6. A Multilayer Modular Organ-on-a-Chip

We designed and created a layered modular organ-on-a-chip system which consists of a reusable acrylic cassette (lid and base) and a layered compressible insert which provides a platform for cell culture and acts as a gasket to seal the system when clamped (FIG. 2A). The resulting geometry consists of two vertically stacked media channels separated by a synthetic track-etched membrane. The insert and cassette can be sterilized with a 70% ethanol solution or UV ozone. The cassette lid has screw clearance holes and an embedded adhered coverslip to enable clamping of the system and channel viewing respectively. Fluid ports within the lid create a connection point for a perfusion pump to perfuse media through both the top and bottom channels as in a traditional PDMS-glass OOC systems. The lid was laser cut to create space for the glass and adhesive layers, screw clearance holes, fluid ports, and insert. The cassette base has threaded holes to accept the screws (FIG. 2A).

The insert geometry can be designed with varying channel lengths and widths (W) for each desired application, while the channel height (H) is defined by the thickness of the silicone (FIG. 2B). Option 1 demonstrates equal channel heights. Option 2 demonstrates an upper channel height less than the lower channel. This can also be reversed depending on experimental needs. Option 3 shows this reversed height configuration while also demonstrating the channel width, W, can be different between channels as well.

Standard Transwell culture is often used when understanding transport or barrier properties of a cell layer. Various cell layers may be formed in the layered modular organ-on-a-chip system to mimic organs and tissues such as blood brain barrier, eye, upper and lower airways (e.g., nasal passages, trachea, and alveoli), gastrointestinal (GI) tract (e.g., stomach, small intestine, large intestine, and rectum), skin/wounds, immune tissues (e.g., lymph nodes, pyer's patches, tonsils, and spleen), heart, liver, pancreas, and engineered tissues (e.g., vascular beds, and organoids).

Compared to standard photolithography techniques, fabrication of both the cassette and the insert may be done with less toxic reagents, inexpensive equipment and materials, and in a cleanroom-free setting, which allows this system to be used and fabricated more easily in non-specialty labs. While no expensive specialty equipment is used to fabricate the modular system, equipment that is commonly available in biology, chemistry, and engineering laboratories, like chemical safety cabinets, laminar flow hoods, and biosafety cabinets, are used.

Additionally, the cassette housing may be reconfigured to improve the insert region of interest (ROI) working distance to the microscope objective. This may be done still by rapid prototyping and laminating materials. Using thin sheets will increase bending under compressive loads, but lamination remedies this by providing structure and reduces any bending when an insert is placed and sealed. Laser cutting a recess in the cassette lid requires cleanup that is otherwise eliminated via thin sheet lamination, and may prove faster and more reliable to manufacture, while also bringing the insert ROI closer to the objective. Overall, this is a viable tool to study cell and drug transport across cellular barriers and how shear stress impacts cell function.

### Example 7. Kidney Model

A kidney model was prepared with an epithelium and an endothelium on a scaffold made of 0.1% Gelatin membrane coating (FIGS. 5A-6E). The epithelium was formed by Madin-Darby canine kidney (MDCK) at 160,000 cells/insert) with a 4-hour adherence time. The endothelium was formed by HUVECs at 150,000 cells/insert with a 4-hour adherence time.

We cultured HUVECs or MDCKs on either side of an insert (FIGS. 5A-5F). Monolayers of MDCKs and HUVECs were on either side of the insert (FIG. 5D) and complete monolayers were through the entire channel (FIGS. 5E-F).

After monolayer formation, the insert can be clamped into the cassette to establish fluid flow and shear stress. To validate that the cells can withstand shear in the cassette and appropriate phenotype shifts are seen, we cultured the HUVEC channel under 3 mL/min media perfusion for 48 hrs (FIG. 6A). Both HUVECs and MDCKs were able to be stably cultured in the cassette and with fluid flow applied to one channel (FIGS. 6B-C) and they were able to maintain complete culture of the entire channel (FIGS. 6D-E).

### Example 8. Placenta Model

A placenta model may be prepared with two or three of an epithelium, a stroma and an endothelium on or in a scaffold having a top scaffold surface and a bottom scaffold surface.

Where the scaffold is a semi-permeable membrane or vitrified membrane, the model may comprise first cells attached to the top scaffold surface and second cells attached to the bottom scaffold surface. Where the scaffold is a hydrated gel, the model may comprise first cells in the scaffold, second cells attached to the top scaffold surface, and third cells attached to the bottom scaffold surface.

The scaffold may be (a) a semi-permeable membrane coated with 0.5 mg/mL fibronectin or 0.1% gelatin at 10-20 μL per side, (b) Collagen-I or decellularized placental hydrated ECM gel, or (c) Collagen-I vitrified membrane.

The epithelium may be formed by BeWo b 30 cells at 100,000 cells/insert with a 4-hour adherence time.

The stroma may be formed by NIH-3T3 or human villous mesenchymal fibroblasts seeded in a pre-gel solution at 1.4×10⁶ cells/mL. The pre-gel solution may comprise collagen-I or decellularized placental or other tissue ECM.

The endothelium may be formed by human umbilical vein endothelial cells (HUVECs) or human placental vascular endothelial cells (HPVECs) at 100,000 cells/insert with a 4-hour adherence time.

In one example, the scaffold is a hydrated gel comprising collagen-I, the epithelium, made with placental epithelial cells (BeWo), is on top of the gel and the endothelium, made from HUVECs, is on the bottom of the gel (FIG. 7).

### Example 9. Cervix Model Scaffold

A cervix model may be prepared with an epithelium and a stroma on different sides of a scaffold.

The scaffold may be (a) a 0.5 mg/mL fibronectin semi-permeable membrane coating or (b) a collagen-I hydrated ECM gel.

The epithelium may be formed by A2EN endocervical cells at 2×10⁵ cells/insert with a 30-minute adherence time.

The stroma may be formed by BJ Fibroblasts at 2×10⁵ cells/insert with a 30-minute adherence time.

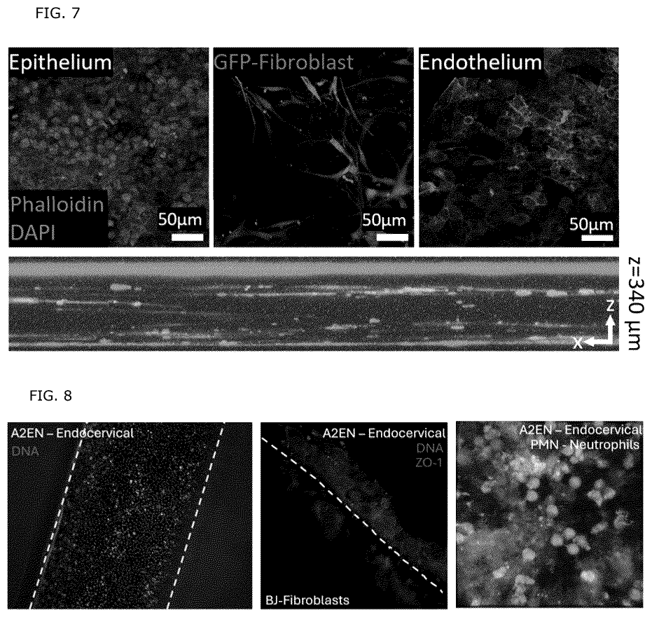

In one example, the scaffold in the insert was a semi-permeable membrane with a fibronectin coating, the A2EN endocervical cells formed the epithelium on one side of the insert while the BJ fibroblasts formed the endothelium on the other side of the insert (FIG. 8).

### Example 10. Vagina Model

A vagina model may be prepared with an epithelium and a stroma on different sides of a scaffold.

The scaffold may be a 0.5 mg/mL fibronectin membrane coating. The epithelium may be formed by VK2 cells at 2×10⁵ cells/insert with a 30-minute adherence time. The stroma may be formed by BJ Fibroblasts at 2×10⁵ cells/insert with a 30-minute adherence time. The cells may be cultured with or without microbiome. The microbiome may be isolated or reconstructed vaginal microbial communities.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A system for culturing cells, comprising:
(a) a cassette comprising a lid and a base, wherein the lid comprises a top channel inlet, a top channel outlet, a bottom channel inlet and a bottom channel outlet;
(b) an insert in the cassette, wherein the insert is between the lid and the base, wherein the insert comprises a top layer having a top channel, a bottom layer having a bottom channel, and a cell culture platform having a top platform surface in contact with the top channel and a bottom platform surface in contact with the bottom channel, wherein the cell culture platform is selected from the group consisting of a vitrified membrane, a hydrated gel, a semi-permeable membrane, and a combination thereof, wherein the top channel is sealed by the lid and the top platform surface and connected with the top channel inlet and the top channel outlet, wherein a top flowing fluid moves to create a wall shear stress in the top channel of 0.25-75 dyne/cm² through the top channel inlet, the top channel, and the top channel outlet; wherein the bottom channel is sealed by the base and the bottom platform surface, and connected with the bottom channel inlet and the bottom channel outlet, wherein a bottom flowing fluid moves to create a wall shear stress in the bottom channel of 0.25-75 dyne/cm² through the bottom channel inlet, the bottom channel, and the bottom channel outlet, and
(c) first cultured cells, wherein the first cultured cells are attached to the top platform surface and exposed directly to the top flowing fluid, are attached to the bottom platform surface and exposed directly to the bottom flowing fluid, or are in the cell culture platform and exposed indirectly to the top flowing fluid and indirectly to the bottom flowing fluid.

2. The system of claim 1, wherein the vitrified membrane has a thickness of 2-50 μm.

3. The system of claim 1, wherein the culture platform consists of the vitrified membrane.

4. The system of claim 3, further comprising second cultured cells.

5. The system of claim 4, wherein the vitrified membrane comprises collagen-I, the first cultured cells are BeWo b30 cells, and the second cultured cells are HUVECs.

6. The system of claim 1, wherein the hydrated gel has a thickness of 50-1500 μm.

7. The system of claim 1, wherein the cell culture platform consists of the hydrated gel.

8. The system of claim 7, further comprising second cultured cells.

9. The system of claim 8, further comprising third cultured cells.

10. The system of claim 9, wherein the hydrated gel is a hydrogel comprising collagen-I or decellularized placental extracellular matrix (ECM), and wherein the first cultured cells are BJ fibroblasts, the second cultured cells are BeWo cells, and the third cultured cells are Human umbilical vein endothelial cells (HUVECs).

11. The system of claim 1, wherein the cell culture platform consists of the semi-permeable membrane having a first semi-permeable membrane surface and a second semi-permeable membrane surface, wherein the first culture cells are attached to the first semi-permeable membrane surface.

12. The system of claim 11, further comprising second cultured cells attached to the second semi-permeable membrane surface.

13. The system of claim 1, wherein the culture platform consists of the hydrated gel having a first gel surface and a second gel surface and the semi-permeable membrane having a first semi-permeable membrane surface and a second semi-permeable membrane surface, wherein the second gel surface and the first semi-permeable membrane surface are in direct contact.

14. The system of claim 13, further comprising second cultured cells.

15. The system of claim 13, further comprising third cultured cells.

16. The system of claim 1, wherein the vitrified membrane comprises first ECM proteins and the first cultured cells are attached to a surface of the vitrified membrane.

17. The system of claim 1, wherein the hydrated gel comprises second ECM proteins and the first cultured cells are in the hydrated gel.

18. The system of claim 1, wherein the semi-permeable membrane has a semi-permeable membrane surface coated with third ECM proteins and the first cultured cells are attached to the coated semi-permeable membrane surface.

19-24. (canceled)

25. The system of claim 1, wherein the top flowing fluid comprises a top agent selected from the group consisting of media, cells, blood, microbiota, pharmacological agents, normoxic air, anoxic air and a combination.

26. The system of claim 25, wherein the top agent migrates from the top channel into the cell culture platform.

27. The system of claim 25, wherein the top agent migrates from the top fluidic channel through the cell culture platform and into the bottom channel.

28. The system of claim 1, wherein the bottom flowing fluid comprises a bottom agent selected from the group consisting of media, cells, blood, microbiota, pharmacological agents, normoxic air, anoxic air and a combination.

29. The system of claim 28, wherein the bottom agent migrates from the top channel into the cell culture platform.

30. The system of claim 28, wherein the bottom agent migrates from the top fluidic channel through the cell culture platform and into the bottom channel.

31-35. (canceled)

36. A method for preparing a system for culturing cells, comprising:
(a) fabricating a cassette with a lid and a base, wherein the lid comprises a top channel inlet, a top channel outlet, a bottom channel inlet and a bottom channel outlet;
(b) providing a cell culture platform having a top platform surface and a bottom platform surface, wherein the cell culture platform is selected from the group consisting of a vitrified membrane comprising first extracellular matrix (ECM) proteins, a hydrated gel comprising second ECM proteins, a semi-permeable membrane, and a combination thereof;
(c) assembling an insert comprising the cell culture platform, a top layer having a top channel in contact with the top platform surface, and a bottom layer having a bottom channel in contact with the bottom platform surface;
(d) placing the insert in the cassette, wherein the insert is between the lid and the base, whereby the top channel is sealed by the lid and the top platform surface and connected with the top channel inlet and the top channel outlet, and the bottom channel is sealed by the base and the bottom platform and connected with the bottom channel inlet and the bottom channel outlet;
(e) perfusing a top flowing fluid at a top channel wall shear stress of 0.25-75 dyne/cm² through the top channel inlet, the top channel, and the top channel outlet; and
(f) perfusing a bottom flowing fluid at a bottom channel wall shear stress 0.25-75 dyne/cm² through the bottom channel inlet, the bottom channel, and the bottom channel outlet.

37-40. (canceled)
